(19) United States
(12) Patent Application Publication
(10) Pub. No.: US 2003/0103959 A1
(43) Pub. Date: Jun. 5, 2003

(54) Methods of providing neuroprotection and/or neurorestoration via the neural activin type IIB receptor

(76) Inventors: Paul E. Hughes, Auckland (NZ); John Anthony Cajetan Fernandez, Auckland (NZ); Sumit Raniga, Auckland (NZ)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/177,735

(22) Filed: Jun. 20, 2002

Related U.S. Application Data
(60) Provisional application No. 60/300,514, filed on Jun. 22, 2001.

(51) Int. Cl. A61K 38/48

(52) U.S. Cl. 424/94.63

(57) ABSTRACT

The neural Type IIB activin receptor (ActRIIB) is strongly expressed in the injured brain, in contrast to the near absence of its expression in the uninjured, non-diseased brain. This invention is directed to the use of ActRIIB activating agents and disinhibiting agents in treating neuronal insult (including stroke). This invention provides agents and methods of using agents that activate or disinhibit ActRIIB, and associated intracellular signaling pathways, as neuroprotective and neurorestorative agents that are specifically targeted to the injured brain and selectively to injured regions of the brain.

### CROSS REFERENCE TO RELATED APPLICATIONS

This application claims the priority under 35 USC 119(e) of Provisional Application No. 60/300,514, filed Jun. 22, 2001, which is incorporated into this application by reference.

### FIELD OF THE INVENTION

The invention is directed to methods and agents for neuroprotection and for neurorestoration of neural tissue following neural disease and injury, including stroke and neurological diseases associated with aging. In particular, the invention is directed to agents and methods of using agents that activate or disinhibit the neural activin type IIB receptor (ActRIIB) and associated intracellular signaling pathways.

### BACKGROUND

Activin is a member of the transforming growth factors-β (TGF-β) superfamily of growth and differentiation factors, which includes glial cell line-derived neurotrophic factor (GDNF) and the bone morphogenetic proteins (BMPs). Originally identified from follicular fluid, activin has been shown to regulate many biological functions in both reproductive and non-reproductive tissue and is currently attracting interest as a potential neurotrophic factor. Classically neurotrophic factors are large protein molecules that support the survival and enhance the function of neurons. In vitro, activin promotes the survival and regulates the phenotype of cultured sympathetic neurons (Fann, M. J. and Patterson, P. H. (1994) Neuropoietic cytokines and activin A differentially regulate the phenotype of cultured sympathetic neurons. Proceedings of the National Academy of Sciences of the United States of America 91, 43-7) as well as exerting neuroprotective effects on dopaminergic neurons damaged by toxins (Krieglstein, K., Suter-Crazzolara, C., Fischer, W. H. and Unsicker, K. (1995) TGF-beta superfamily members promote survival of midbrain dopaminergic neurons and protect them against MPP+ toxicity. EMBO Journal 14, 736-42). In vivo studies have shown that activin-A is increased both at the mRNA and protein level after brain injuries with little expression of activin functional antagonists. (Lai, M., Gluckman, P., Dragunow, M. and Hughes, P. E. (1997) Focal brain injury increases activin betaA mRNA expression in hippocampal neurons. Neuroreport 8, 2691-4; and data contained this application.) Furthermore, a recent study demonstrates that administration of recombinant human (rh) activin-A has potent neurotrophic effects in the quinolinic acid (QA) rat lesion model of Huntington's Disease (HD) (Hughes, P. E., Alexi, T., Williams, C. E., Clark, R. G. and Gluckman, P. D. (1999) Administration of recombinant human Activin-A has powerful neurotrophic effects on select striatal phenotypes in the quinolinic acid lesion model of Huntington's disease. Neuroscience 92, 197-209) and that the neuroprotective effects of basic fibroblast growth factor (bFGF) are mediated by activin-A in vivo (Mattson, M. P. (2000) Activin to the rescue for overexcited neurons. Nature Medicine 6, 739-741; Tretter Y P, H. M., Munz B, Bruggencate G T, Werner S & Alzheimer C (2000) Induction of activin A is essential for the neuroprotective action of basic fibroblast growth factor in vivo. Nature Medicine 6, 812-815). The transient developmental expression of activin, and in particular the localization of activin receptors to widespread regions in the adult brain (Bengtsson, H., Soderstrom, S. and Ebendal, T. (1995) Expression of activin receptors type I and II only partially overlaps in the nervous system. Neuroreport 7, 113-6) further suggests an important role for activin receptors in the central nervous system (CNS).

Activin interacts indirectly and directly with two different types of receptors, type I and type II respectively, both containing an extracellular domain, a single transmembrane region and a large intracellular domain that includes a serine/threonine kinase domain. There are two type II ligand binding receptors, activin receptor type IIA (ActRIIA) and activin receptor type IIB (ActRIIB), and two type I signal transducing receptors, activin receptor-like kinase-2 (Alk-2) and activin receptor-like kinase-4 (Alk-4) that are suggested to be involved in activin signaling.

Briefly, activin binds to the type II receptor, whose serine/threonine kinase is constitutively active. This complex recruits a type I receptor. The type II receptor phosphorylates the type I receptor. This activates the serine/threonine kinase of the type I receptor. Both Smad2 and Smad3 (mammalian homologs of the insect factor “mothers against dpp”), which are transcription factors involved in TGF-β family signaling, interact with the activin receptor complex upon activin stimulation, most probably by functionally interacting with ActRI (Zhang, Y., Feng, X., We, R. and Derynck, R. (1996) Receptor-associated Mad homologues synergize as effectors of the TGF-beta response. Nature 383, 168-72; Nakao, A., Imamura, T., Souchelnytskyi, S., Kawabata, M., Ishisaki, A., Oeda, E., Tamaki, K., Hanai, J., Heldin, C. H., Miyazono, K. and ten Dijke, P. (1997) TGF-beta receptor-mediated signaling through Smad2, Smad3 and Smad4. EMBO Journal 16, 5353-62; Lebrun, J. J., Takabe, K., Chen, Y. and Vale, W. (1999) Roles of pathway-specific and inhibitory Smads in activin receptor signaling. Molecular Endocrinology 13, 15-23). The type I receptor phosphorylates the Smads (Abdollah, S., Macias-Silva, M., Tsukazaki, T., Hayashi, H., Attisano, L. and Wrana, J. L. (1997) TbetaRI phosphorylation of Smad2 on Ser465 and Ser467 is required for Smad2-Smad4 complex formation and signaling. Journal of Biological Chemistry 272, 27678-85). Smad2 and Smad3 are early intracellular downstream components of activin receptor signal transduction. Once phosphorylated these two Smads are released into the cytoplasm where they interact with the common-partner Smad4 (Nakao, A., Imamura, T., Souchelnytskyi, S., Kawabata, M., Ishisaki, A., Oeda, E., Tamaki, K., Hanai, J., Heldin, C. H., Miyazono, K. and ten Dijke, P. (1997) TGF-beta receptor-mediated signaling through Smad2, Smad3 and Smad4. EMBO Journal 16, 5353-62).

All documents cited in this application are incorporated into this application by reference.

### SUMMARY OF THE INVENTION

In a first aspect, this invention provides a method of treating a mammal to rescue neurons otherwise destined to die, or to restore the phenotype and function of neurons degenerating, as the result of neuronal insult, comprising increasing within the mammal the concentration of an neural activin type IIB receptor-activating agent effective to rescue neurons otherwise destined to die, or to restore the phenotype and function of neurons degenerating, as the result of the neuronal insult. Typically, this method comprises adiminstering to the mammal a therapeutic amount of a neural activin type IIB receptor-activating agent. It may also comprise administration of two or more neural activin type IIB receptor-activating agents, such as activin and a non-activin neural activin type IIB receptor-activating agent; or a neural activin type IIB receptor-activating agent and an agent that decreases the active concentration of neural activin type IIB receptor-inhibiting agents within the mammal (an activin type IIB receptor disinhibitor).

In a second aspect, this invention provides a method of treating a mammal to rescue neurons otherwise destined to die, or to restore the phenotype and function of neurons degenerating, as the result of neuronal insult, comprising decreasing the active concentration of neural activin type IIB receptor-inhibiting agents within the mammal to a level effective to rescue neurons otherwise destined to die, or to restore the phenotype and function of neurons degenerating, as the result of the neuronal insult.

In a third aspect, this invention provides a method of treating a mammal to rescue neurons otherwise destined to die, or to restore the phenotype and function of neurons degenerating, as the result of neuronal insult, comprising increasing within the mammal the concentration of an agent that relieves inhibition of the neural activin type IIB receptor (a neural activin type IIB receptor disinhibitor) effective to activate the neural activin type IIB receptor to rescue neurons otherwise destined to die, or to restore the phenotype and function of neurons degenerating, as the result of the neuronal insult. Typically, this method comprises administering to the mammal a therapeutic amount of a neural activin type IIB receptor-disinhibitor.

In further aspects, this invention provides medicaments suitable for the practice of the methods of the first three aspects of the invention, and methods of making such medicaments.

### DESCRIPTION OF THE INVENTION

This invention is directed to new therapeutic uses of agents that confer neuroprotective or neurorestorative responses due to their actions to activate or to disinhibit the neural activin type IIB receptor (ActRIIB) and associated intracellular signaling pathways. Specifically, this invention is directed to the use of these agents in neurological insult where ActRIIB is highly expressed. The strong expression of ActRIIB in the injured brain is in contrast to its near absence in the uninjured, non-diseased brain. This invention thus represents a novel mechanism whereby a neuroprotective and neurorestorative agent could be administered to a mammal and the effects of neuroprotection and neurorestoration are specifically targeted to 1. the injured brain and 2. selectively injured regions.

These involve one or more of:

1. administering to the mammal activin or an analog thereof to activate ActRIIB;

2. increasing the active concentration of activin in a mammal using various means thereof to activate ActRIIB;

3. administering to the mammal other agents which activate ActRIIB (ActRIIB activators);

4. increasing the active concentration of other agents which activate ActRIIB (ActRIIB activators);

5. administering to the mammal other agents which activate ActRIIB indirectly via dis-inhibition (ActRIIB disinhibitors);

6. increasing the active concentration of other agents which activate ActRIIB indirectly via dis-inhibition (ActRIIB disinhibitors);

7. administering to the mammal activin or an analog thereof in combination with administering another agent which can either (a) activate ActRIIB (an ActRIIB activator) or (b) activate ActRIIB indirectly via dis-inhibition (an ActRIIB disinhibitor) to further activate ActRIIB; or

8. increasing the active concentration of activin in a mammal using various means thereof in combination with administering another agent which can either (a) activate ActRIIB (an ActRIIB activator) or (b) activate ActRIIB indirectly via dis-inhibition (an ActRIIB disinhibitor) to further activate ActRIIB.

The approaches of the invention have application in the treatment of mammals who have suffered neuronal insult. Mammals suffering from neuronal insult will benefit greatly by a treatment protocol able to rescue damaged and dying neuronal cell populations.

In the brain the expression of Alk-2 and Alk-4 mRNAs is constitutively high throughout the entire brain and ActRIIB mRNA is significantly expressed in the amygdala and hippocampus. In contrast, ActRIIB mRNA expression is constitutively low in the uninjured brain. With unilateral brain ligation injury in rats we have found dramatic and persistent up-regulation of ActRIIB mRNA in brain tissue in the injured hemisphere. Alk-2 mRNA showed a transient and modest up-regulation on the ligated side and a dramatic increase on the non-ligated side. The expression of ActRIIA and Alk-4 mRNA was found to decrease on the injured side after HI.

Therefore, the prior observed neuroprotective and neurorestorative role of activin is mediated via the neural activin type IIB receptor (ActRIIB) and associated downstream and intracellular signaling pathways. Specifically contemplated analogs, activators, and disinhibitors related to this invention are therefore those that bind either directly or indirectly to and activate or disinhibit the ActRIIB so that intracellular signaling via neuroprotective and neurorestorative activin pathways is increased in injured or diseased or non-functioning neurons.

Agents which act on the neural activin type IIB receptor to induce intracellular signaling via Smads will be neuroprotective, neurorestorative, and will positively regulate the phenotype and functioning of injured neurons, and that the effects of these agents will be selective for the injured brain. Further, since activin is clearly neuroprotective when administered exogenously (Hughes, P. E., Alexi, T., Williams, C. E., Clark, R. G. and Gluckman, P. D. (1999) Administration of recombinant human Activin-A has powerful neurotrophic effects on select striatal phenotypes in the quinolinic acid lesion model of Huntington's disease. Neuroscience 92, 197-209) and since high levels of both activin protein and signaling via ActRIIB using methods discussed in this applicaton will prove an especially powerful neuroprotective and neurorestorative effect subsequent to injury.

As broadly defined above, the present invention relates primarily to neuroprotection and neurorestoration provided by agents which act via the neural activin type IIB receptor (ActRIIB). Neuroprotection is the maintenance of neuronal cells which would otherwise be destined to die as a result of a prior neuronal insult or neurodegenerative disease. Neurorestoration is the restoration of cells that are not dead but exist in a seriously dysfunctional state. Atrophied cholinergic and cortical neurons in the early stages of Alzheimer's disease represent one, but not a limiting example of cells that are not dead yet but are seriously dysfunctional. The invention relates both to phenotype and functional restoration. Phenotype relates to key enzymes produced and required for functioning of the neuron. Thus phenotypic and functional restoration are intimately linked. Degenerating neuronal cells which have lost their phenotype as the result of a prior neuronal insult can be phenotypically restored by activin. Agents which act positively on ActRIIB will have the same effect after brain injury.

Neuroprotection/neurorestoration mediated by ActRIIB is able to be effected using two approaches, the first by increasing the concentration of activin able to act at ActRIIB, and the second by activating or increasing the action of ActRIIB with agents other than activin. The first approach builds upon findings that increasing the effective concentration of activin within a mammal following neuronal insult rescues neurons and/or restores their phenotype. The second approach focuses upon activating the neural type IIB activin receptor as defined above through the use of small molecule agents which can either further promote (ActRIIB activators) or disinhibit (ActRIIB disinhibitors) activin signaling through this receptor.

The first approach is through a focus upon activin. The applicants have found that increasing the effective concentration of activin within a mammal following neuronal insult rescues neurons and/or restores their phenotype (Hughes, P. E., Alexi, T., Williams, C. E., Clark, R. G. and Gluckman, P. D. (1999) Administration of recombinant human activin-A has powerful neurotrophic effects on select striatal phenotypes in the quinolinic acid lesion model of Huntington's disease. Neuroscience 92, 197-209). Activin itself is critical to this approach. There are three isoforms of activin, designated activin A, activin B and activin AB. Structural analysis shows that activins are disulphide linked dimers of two subunits, which are two distinct 14 kDa β subunits (βA and βB) (Ying S. Y. (1989). Inhibins, activins and follistatins. Journal of Steroid Biochemistry, 33:705-713; Vale W., Hsueh A. J. W., River C. and Yu A. (1990). The inhibin/activin family of hormones and growth factors. In Peptide Growth Factors and their Receptors, Handbook of Experimental Pharmacology. M Sporn and A B Roberts, eds. (Berlin: Springer-Verlag), pp 211-248). Activins (28 kDa) are homodimers of the two β subunits. The mature βA or βB subunit has 116 or 115 amino acids respectively including 9 cysteines with no glycosylation sites. The two β subunits share about 85% homology within each species, and are also highly homologous across species. The mature βA subunits are completely identical across porcine, bovine, human and murine species and the mature ββB subunit only has differences at four amino acid positions (Esch F. S., Shimasaki S., Mercado M., Cooksey K., Ling N., Ying S., Ueno N. X., and Guillemin R. (1987). Structural characterization of follistatin: a novel follicle-stimulating hormone release-inhibiting polypeptide from the gonad. Molecular Endocrinology, 1(11):849-855).

Recently, the molecular cloning of activin βC, βD, and βE subunits has been reported (Fang J. M., Yin W. S., Smiley E., Wang S. Q., and Bonadio J. (1996). Molecular cloning of the mouse activin beta(E) subunit gene. Biochemical and Biophysical Research Communications, 228(3):669-674). Activins made up from or including one or more of these subunits are suitable for the practice of the invention.

All of the above forms of activin are contemplated for use in this invention.

“Activin” as used herein means activin A, activin B or activin AB of mammalian origin and preferably of human, porcine, bovine or murine origin. The preferred form of activin for use in this invention is activin A. This is available from National Institutes of Health, USA.

It is also contemplated that activin analogs can be employed in this invention. As used herein, “analog” means a protein which is a variant of activin through insertion, deletion or substitution of one or more amino acids but which retains at least substantial functional equivalency.

A protein is a functional equivalent of another protein for a specific function if the equivalent protein is immunologically cross-reactive with, and has at least substantially the same function as, the original protein. The equivalent can be, for example, a fragment of the protein, a fusion of the protein with another protein or carrier, or a fusion of a fragment with additional amino acids. For example, it is possible to substitute amino acids in a sequence with equivalent amino acids using conventional techniques. Groups of amino acids normally held to be equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp.

Functional equivalency of activin analogs can also be readily screened for by reference to the ability of the analog to both bind to and activate the appropriate receptor. In this case, the receptor is an activin type IIB receptor.

“ActRIIB activators” or “neural activin type IIB receptor-activating agents” is used herein to mean small molecules that act as ActRIIB agonists directly at the ActRIIB receptor molecule. Such stimulatory ligands can be identified by various screening protocols employing cultured neuron-like cell lines (for example using hNT cells, Ren, R. F., Hawver, D. B., Kim, R. S. and Flanders, K. C. (1997) Transforming growth factory-β protects human hNT cells from degeneration induced by β-amyloid peptide: involvement of the TGF-β type II receptor. Mol. Brain Res. 48, 315-322) and measurement of ActRIIB-mediated signal transduction via Smad phosphorylation. Further classical “grind and bind” ligand-binding experiments can also be utilized. Here, whole brain or specific brain regions would be homogenized and the specific-binding of novel compounds to ActRIIB characterized. This technique allows further characterization of specificity and affinity (potency) of the compound for ActRIIB.

“ActRIIB disinhibitors” or “agents that relieve inhibition of the neural activin type IIB receptor” is used herein to mean small molecules that act to disinhibit signaling through ActRIIB. These include but are not limited to FKBP12 antagonists including the immunosuppressant drugs (including cyclosporin A, rapamycin, tacrolimus (FK-506), ascomycin, GPI-1046 (3-pyridin-3-ylpropyl 1-(3,3-dimethyl-2-oxopentanoyl)-pyrrolidine-2-carboxylate), N-Me-Val-4-cyclosporin A) and non-immunosuppressant orally bioavailable drugs such as V-10,367 (1-(3-phenylpropyl)-4-pyridin-3-ylbutyl 1-[2-oxo-2-(3,4,5-trimethoxyphenyl)acetyl]piperidine-2-carboxylate), small molecules that disrupt TRAP-1 binding to neural type I activin receptors, small molecule antagonists of the mammalian version of the pseudoreceptor BAMBI, and agents which modulate endoglin and betaglycan function.

“Neuronal insult” is used herein in its broadest possible sense and includes neuronal insults due to trauma (neural injury), toxins, asphyxia, hypoxic-ischemic injury (HI, stroke), and progressive neurodegenerative diseases including Huntington's disease, Alzheimer's disease, Parkinson's disease, multiple sclerosis, amyotrophic lateral sclerosis, Lewy body diseases, and peripheral neuropathy (including diabetes-related peripheral neuropathy, drug-induced peripheral neuropathy, and all other forms of peripheral neuropathy).

A “therapeutically effective amount” is an amount sufficient to provide the desired action, or effect, such as to treat a mammal by rescuing neurons otherwise destined to die, or by restoring the phenotype and function of neurons degenerating, as a result of a prior neuronal insult.

Most conveniently, the effective concentration of activin is increased through direct administration using either activin itself or an activin prodrug (a form which is cleaved within the body to release activin). In addition, the effective concentration of activin may be increased through administration of either activin agonists or inhibitors of activin antagonists. Actvin agonists and inhibitors of activin antagonists may be administered once or twice per day intraperitoneally, subcutaneously, intravenously or orally in doses in the range of about 1 μg/kg to about 500 mg/kg. Activin agonists are substances which effect a direct increase in production or activity of activin within the body, e.g. basic fibroblast growth factor (bFGF), follicular stimulating hormone (FSH), cyclic adenosine monophosphate (cAMP), 12-O-tetradecanoylphorbol 13-acetate (TPA), transforming growth factor-β (TGF-β), interleukin-1β (IL-1β) and tumor necrosis factor-α (TNF-α). Inhibitors of activin antagonists are compounds that inhibit the action of substances which bind activin or otherwise prevent or reduce the action of activin within the body. These latter compounds exert an indirect effect on effective activin concentrations through the removal of an inhibitory mechanism, and include substances such as estradiol.

Follistatin is one such substance. It is a single chain glycosylated protein, which was first isolated from porcine and bovine follicular fluid. The amino acid sequence of follistatin is distinct from those of the activin subunits and any other proteins in the TGF-β family. However, across species, follistatin amino acid sequence is highly conserved with over 98% homology.

As a binding protein for activin, follistatin has been observed to have different actions on the biological activities of activin. Follistatin can directly bind to activin to neutralize its function in many systems (Mathews, L. S. (1994) Activin receptors and cellular signaling by the receptor serine kinase family. Endocrine Reviews 15, 310-25). However, it has also been suggested to have an ability to enhance activin action through either bringing activin to its receptors or maintaining a high local concentration of activin. Thus follistatin may exert a dual effect in mediating activin activities (Macconell, L. A., Barth, S. and Roberts, V. J. (1996) Distribution of follistatin messenger ribonucleic acid in the rat brain: implications for a role in the regulation of central reproductive functions. Endocrinology 137, 2150-8) as both agonist and antagonist.

Inhibin is also an activin antagonist. The mechanism by which this is achieved is not completely understood but is likely to include competitive binding to the activin receptor. Therefore, effecting a decrease in the production or action of inhibin is able to increase the effective concentration of activin.

Another possibility is administration of a replicable vehicle encoding activin to the mammal. Such a vehicle (which may be a modified cell line or virus which expresses activin within the mammal) could have application in increasing the concentration of activin within the mammal for a prolonged period.

Similar to TGF-β receptors, there are two types of activin receptors, termed activin type I receptor (ActRI) and activin type II receptor (ActRII). ActRIIA was the first receptor identified for activin and for other members in the TGF-β superfamily (Matthews L. S., and Vale W. W. (1991). Expression cloning of an activin receptor, a predicted transmembrane serine kinase. Cell, 65:973-982). The mature ActRIIA is comprised of 494 amino acids which includes a small 116 amino acid extracellular ligand binding domain, a single transmembrane domain and an intracellular serine/threonine kinase domain, which is common in the TGF-β superfamily. Over 90% sequence homology of ActRIIA has been observed across species, which is consistent to the high similarity of mature activin βA sequences in various species (Mathews, L. S. (1994) Activin receptors and cellular signaling by the receptor serine kinase family. Endocrine Reviews 15, 310-25). A distinct but closely related activin receptor ActRIIB and its four isoforms have subsequently been characterized (Mathews, L. S. (1994) Activin receptors and cellular signaling by the receptor serine kinase family. Endocrine Reviews 15, 310-25; Matthews L. S., Vale W. W., and Kinter C. R. (1992). Cloning of a second type of activin receptor and functional characterization in Xenopus embryos. Science, 255:1702-1705; Attisano L., Wrana J. L., Cheifet S., and Massague J. (1992). Novel activin receptors: distinct genes and alternative mRNA splicing generate a repertoire of serine/threonine kinase receptors. Cell, 68:97-108). ActRIIA and ActRIIB are approximately 50-60% identical in the ligand-binding domain and 60-70% identical in the kinase domain. ActRIIA, ActRIIB and its isoforms all bind to activin with high affinity.

Collectively, ActRIIA, ActRIIB and their isoforms are referred to herein as “activin type II receptors”.

Activin type II receptors are distinct from Activin type I receptors (ActRI). ActRI and its isoforms have been cloned using PCR with oligonucleotides based on the ActRII sequence. These receptors also have highly conserved serine kinases. However, cells expressing ActRI but not ActRII cannot bind to activin alone (Mathews, L. S. (1994) Activin receptors and cellular signaling by the receptor serine kinase family. Endocrine Reviews 15, 310-25), hence the ability of novel neuroprotective and neurorestorative agents to positively regulate (stimulate) signaling through the neural activin type IIB receptors is considered critical to this invention.

Activin initiates signal transduction across the membrane through both ActRI and ActRIIB which can form a stable complex with the ligand (Mathews, L. S. (1994) Activin receptors and cellular signaling by the receptor serine kinase family. Endocrine Reviews 15, 310-25). ActRIIB binds activin and then associates with a type I receptor. This is followed by auto- and trans-phosphorylation between the two receptors and the initiation of intracellular signaling.

This leads to the second approach to neuroprotection and/or neurorestoration. This approach focuses upon activating the neural activin type IIB receptor as defined above through the use of small molecule agents which can either further promote (ActRIIB activators) or disinhibit (ActRIIB disinhibitors) activin signaling through this receptor. Thus these compounds could potentially be used in combination with administration of activin or activin ligands, or in combination with techniques used to raise neural activin levels within the injured brain to produce potentially synergistic neuroprotective and neurorestorative effects.

Included in this group of small molecules are the ActRIIB activators, small molecules that act as ActRIIB agonists directly at the ActRIIB receptor molecule. Such stimulatory ligands can be identified by various screening protocols employing cultured neuron-like cell lines (for example using hNT cells, Ren, R. F., Hawver, D. B., Kim, R. S. and Flanders, K. C. (1997) Transforming growth factor-β protects human hNT cells from degeneration induced by β-amyloid peptide: involvement of the TGF-β type II receptor. Mol. Brain Res. 48, 315-322) and measurement of ActRIIB receptor-mediated signal transduction via Smad phosphorylation. Further classical “grind and bind” ligand-binding experiments can also be utilized. Here, whole brain or specific brain regions would be homogenized and the specific-binding of novel compounds to the ActRIIB receptor characterized. This technique allows further characterization of specificity and affinity (potency) of the compound for the ActRIIB receptor complex.

For example, primary rat neuronal cells derived from E15-E18 embryos or neural cell lines including but not limited to PC12, Neuro2A and hNT would be plated into 96 well plates to allow high throughput screening of compounds with the aim of identifying compounds with specific activity at the ActRIIB receptor. Immunohistochemistry and PCR would be used to confirm expression of the ActRIIB receptor in cultures. Compounds would be added to cultures and their ability to initiate trans-phosphorylation of ActRIA or ActRIB or downstream phosphorylation of Smads investigated by the following mechanisms:

1. immunoprecipitation of ActRI receptors followed by western blotting of the immunoprecipitated receptors and then utilization of an anti-phospho-serine/threonine antibody to probe the blot for changes in receptor phosphorylation. Antibodies selective for either ActRIA or ActRIB may be used during the immunoprecipitation step to investigate the two receptors separately;

2. immunoprecipitation of Smad proteins followed by western blotting of the immunoprecipitated proteins and then utilization of an anti-phospho-serine/threonine antibody to probe the blot for changes in Smad phosphorylation. Antibodies selective for different Smad family proteins may be used during the immunoprecipitation step to investigate different Smad family members separately;

3. Direct measurement of phospho-Smad expression by immunocytochemistry in cultures using Smad phospho-specific antibodies. For example, an anti-phospho-Smad2 antibody can be purchased from Upstate Biotechnology, Catalog number #06-829. This rabbit IgG antibody is specific for phosphorylated Ser 465/467 residues of human Smad2. It is not clear whether the antibody recognizes a singular phosphorylated site or both phosphorylated residues of Smad2. This antibody can be used for immunoblotting and shows species cross-reactivity against rat, mouse, xenopus and human phosphorylated Smad2.

To determine that the effect is mediated solely by the actions of the compound at ActRIIB and not ActRIIA receptors the effect of addition of increasing doses of truncated ActRIIB receptors into the media can be investigated. The attenuation or reversal of the ability of the compound to initiate trans-phosphorylation of ActRIA or ActRIB or increase downstream phosphorylation of Smads would indicate activity of the compound at the ActRIIB receptor.

Included in the group of small molecules are the ActRIIB disinhibitors, small molecules that act, often indirectly, to disinhibit signaling through the ActRIIB receptor. Included here would be the immunosuppressant drugs (including but not limited to cyclosporin A (0.1-40 mg/kg once or twice per day, administered intraperitoneally, subcutaneously, intravenously or orally); rapamycin (1-500 mg/kg once or twice per day, administered intraperitoneally, subcutaneously, intravenously or orally); tacrolimus (1-5 mg/kg intravenous bolus); ascomycin (1-500 mg/kg once or twice per day, administered intraperitoneally, subcutaneously, intravenously or orally); GPI-1046 (1-100 mg/kg once or twice per day, administered intraperitoneally, subcutaneously, intravenously or orally); N-Me-Val-4-cyclosporin A (0.1-40 mg/kg once or twice per day, administered intraperitoneally, subcutaneously, intravenously or orally); and non-immunosuppressant orally bioavailable drugs (such as V-10,367 (10-500 mg/kg per day, intravenously or orally) (Costantini, L. C., Chaturvedi, P., Armistead, D. M., McCaffrey, P. G., Deacon, T. W., Isacson O. (1998) A novel immunophilin ligand: distinct branching effects on dopaminergic neurons in culture and neurotrophic actions after oral administration in an animal model of Parkinson's disease. Neurobiol Dis 5, 97-106) that act as FKBP12 antagonists (Snyder, S. H., Sabatini, D. M., Lai, M. M., Steiner, J. P., Hamilton, G. S. and Suzdak, P. D. (1998) Neural actions of immunophilin ligands. Trends in Pharmacol Sci. 19, 21-26). The immunophilin FKBP12 is one example of a molecule known to inhibit signaling through ActRIIB by interacting with type I activin receptors in such a fashion as to inhibit successful intracellular signaling leading to phosphorylation of Smad proteins and their subsequent translocation to the nucleus to regulate gene expression (Huse, M., Chen, Y. G., Massague, J. and Kuriyan J. (1999) Crystal structure of the cytoplasmic domain of the type I TGF beta receptor in complex with FKBP12. Cell 96, 425-436; Wang T., Donahoe, P. K. and Zervos, A. S. (1994) Specific interaction of type I receptors of the TGF-β family with the immunophilin FKBP12. Science 265, 674-676). The immunosuppressants are known to bind to FKBP12. In doing so they remove the negative influence that FKBP12 binding has on signaling through ActRIIB receptors and thus act as ActRIIB disinhibitors.

Another group of compounds that are included disinhibit ActRIIB signaling by interacting with the TRAP-1 protein which interacts with the activated/phosphorylated type I receptor to inhibit further signaling (Charng, M. J., Zhang, D., Kinnunen, P. and Schneider, M. D. (1998) A novel protein distinguishes between quiescent and activated forms of the type I transforming growth factor beta receptor. J. Biol. Chem. 273, 9365-9368). Thus small molecules that would disrupt TRAP-1 binding to neural type I activin receptors would also fall into the group of agents called ActRIIB disinhibitors.

Furthermore small molecule antagonists of the mammalian version of the pseudoreceptor BAMBI would also full under this category and are included (Onichtchouk, D., Chen, Y. G., Dosch, R., Gawantka V., Delius H., Massague J. and Niehrs, C. (1999) Silencing of TGF-β signaling by the pseudoreceptor BAMBI. Nature 401, 480-485). Also agents which modulate endoglin and betaglycan function will also fall under this category (Barbara N. P., Wrana J. L. and Letarte, M. (1999) Endoglin is an accessory protein that interacts with the signaling receptor complex of multiple members of the transforming growth factor-beta superfamily. J. Biol. Chem. 274, 584-594). Another group of agents that are included disinhibit ActRIIB signaling by interrupting caveolin-1 (Cav-1) binding to type I receptors (Razani, B., Zhang, X. L., Bitzer, M., von Gersdorff, G., Bottinger, E. P. and Lisanti, M. P. (2001) Caveolin-1 regulates transforming growth factor (TGF)-beta/SMAD signaling through an interaction with the TGF beta type I receptor. J. Biol. Chem. 276, 6727-6738).

For the intended therapeutic application, the active compound(s) (activin, analog, ActRIIB activator, ActRIIB disinhibitor) will be formulated as a medicament. The details of the formulation will ultimately depend upon the insult to be remedied and the route of administration, but will usually include combination of the active compound with a suitable carrier, vehicle or diluent.

In general, the active compound(s) will be administered as pharmaceutical compositions by one of the following routes: directly to the central nervous system, oral, topical, systemic (e.g. transdermal, intranasal, or by suppository), intracerebroventricular, epidural, parenteral (e.g. intramuscular, subcutaneous, intraperitoneal injection, or intravenous injection), by implantation and by infusion through such devices as osmotic pumps, transdermal patches and the like. Compositions may take the form of tablets, pills, capsules, semisolids, powders, sustained release formulation, solutions, suspensions, elixirs, aerosols or any other appropriate compositions; and comprise at least one active compound(s) in combination with at least one pharmaceutically acceptable excipient. Suitable excipients are well known to persons of ordinary skill in the art, and they, and the methods of formulating the compositions, may be found in such standard references as Gennaro, ed., Remington: The Science and Practice of Pharmacy, 20th ed., Lippincott, Williams & Wilkins, 2000. Suitable liquid carriers, especially for injectable solutions, include water, aqueous saline solution, aqueous dextrose solution, and the like, with isotonic solutions being preferred for intravenous administration.

The active compound(s) can be administered directly to the central nervous system. This route of administration can involve, for example, lateral cerebroventricular injection, focal injection, or a surgically inserted shunt into the lateral cerebral ventricle.

Desirably, if possible, the active compound(s) will be administered orally. The amount of the active compound(s) in the composition may vary widely depending on the type of composition, size of a unit dosage, kind of excipients and other factors well known to those of ordinary skill in the art. In general the final composition may comprise from 0.0001 percent by weight (% w) to 10% w of the active compound(s), preferably 0.001% w to 1% w, with the remainder being the excipient or excipients.

The active compound(s) are also suitably administered by a sustained-release system. Suitable examples of sustained-release compositions include semi-permeable polymer matrices in the form of shaped articles, e.g. films, or macrocapsules. Sustained-release matrices include polylactides (U.S. Pat. No. 3,773,919; EP 0 058 481), copolymers of L-glutamic acid and γ-ethyl-L-glutamate (Sidman et al., Biopolymers, 22, 547-556 (1983)), poly(2-hydroxyethyl methacrylate) (Langer et al., J. Biomed. Mater. Res., 15, 267-277 (1981)), ethylene vinyl acetate (Langer et al., supra), or poly-D-(-)-3-hydroxybutyric acid (EP 0 133 988). Sustained-release compositions also include a liposomally entrapped compound. Liposomes containing the compound are prepared by methods known per se: DE 32 18 121; Hwang et al., Proc. Nat'l Acad. Sci. USA, 77(7), 4030-4034 (1980); EP 0 052 322; EP 0 036 676; EP 0 088 046; EP 0 143 949; EP 0 142 641; JP 83-118008; U.S. Pat Nos. 4,485,045, 4,544,545; and EP 0 102 324. Ordinarily, the liposomes are of the small (from or about 200 to 800 Ångstroms) unilamellar type in which the lipid content is greater than about 30 mole percent cholesterol, the selected proportion being adjusted for the most efficacious therapy.

The active compound(s) may also be PEGylated to increase their lifetime in vivo, based on, e.g. the conjugate technology described in WO 95/32003.

The calculation of the effective amount of the active compound(s) to be administered will be dependent upon the route of administration, the disease or injury, its severity, the age and relative health of the patient being treated, the potency of the compound(s) and other factors, and will be routine to a persons of ordinary skill in the art. For a human, where the dose is administered centrally, a suitable dose range for activin is between about 0.3 μg and about 1000 μg per Kg of body weight per day; a preferred dose range is between about 1 μg/Kg/day and about 300 μg/Kg/day, and a more preferred dose range is from about 3 μg/Kg/day to about 100 μg/Kg/day. For peripheral administration, the doses are about 10-fold to 1000-fold higher; and suitable dose ranges will be readily determinable by comparing the activities of a peripherally administered active compound(s) with the activity of the centrally-administered compound in a suitable model and scaling the central compound dose range above accordingly. Suitable dose ranges for other active compound(s) will be readily determinable by comparing the activities of the compounds with the activity of activin in a suitable model and scaling the activin dose range above accordingly; and suitable dose ranges for prodrugs will be determinable in the same manner. A person of ordinary skill in the art will be able without undue experimentation, having regard to that skill and this disclosure, to determine a therapeutically effective amount of the active compound(s) for a given neuronal insult and mammal to be treated.

If desired, more than one active compound may be administered. In addition, the active compound(s) may be administered in combination with, prior to, or following:

1. other treatments to improve neuronal function; repair neurons or glia; promote neuronal migration; promote neuronal proliferation; prevent apoptosis and necrosis; promote neuromodulation;

2. growth factors or associated derivatives; anti-apoptotic agents; free radical scavengers; free radical synthesis inhibitors; agents to decrease energy depletion; inhibitors of leukocyte, microglial or cytokine effects; inhibitors of glutamate release; blockers of glutamate receptors, and devices for treating hypothermia.

The following examples are some of the many treatment regimes suitable for the practice of the invention. It will however be appreciated that the treatment examples are non-limiting.

### TREATMENT EXAMPLE 1

### Acute Neurological Injury (Stroke).

A patient presents with symptoms indicative of cerebral infarction. Activin in a single bolus dose of 3-300 μg/kg of body weight per day is administered via lateral cerebro-ventricular injection into the brain of the patient to prevent neurodegeneration following cerebral infarction. The preferred dose is 10 μg/kg. Alternatively, an analog of activin with activity at ActRIIB and/or a biologically active activin mimetic with activity at ActRIIB could be administered. Treatment is commenced as soon as possible after the head injury and then for 4 days thereafter.

### TREATMENT EXAMPLE 2

### Acute Neurological Injury (Stroke). Combination Therapy (ActRIIB Activator, for Example Activin, in Combination with an ActRIIB Disinhibitor, for Example the Immunophilin Tacrolimus)

A patient presents with symptoms indicative of cerebral infarction. Activin in a single bolus dose of 3-300 μg/kg of body weight per day is administered via lateral cerebro-ventricular injection into the brain of the patient to prevent neurodegeneration following cerebral infarction. The preferred dose is 10 μg/kg. Alternatively, an analog of activin with activity at ActRIIB and/or a biologically active activin mimetic with activity at ActRIIB could be administered. Treatment is commenced as soon as possible after the head injury and then each day for 4 days thereafter. The patient also receives tacrolimus intravenously each day at a dose of 0.1-10 mg/kg, 15 minutes before the bolus dose of activin is administered. The preferred dose of tacrolimus is 1 mg/kg iv.

### TREATMENT EXAMPLE 3

### Treatment of a Chronic Neurodegenerative Disease, for Example Alzheimer's Disease.

A patient presents with symptoms indicative of Alzheimer's disease. An orally available ActRIIB agonist with neuronal penetrance is administered by mouth at a dose of 0.1 μg-10 mg/kg of body weight per day to the patient to prevent Alzheimer's associated neurodegeneration. Mechanisms used to identify small molecule, orally bioavailable ActRIIB agonists with neuronal penetrance have been mentioned previously. Treatment is commenced as soon as possible and frequently enough to maintain neuroprotective blood levels as would be determined.

### TREATMENT EXAMPLE 4

### Treatment of a Chronic Neurodegenerative Disease, for Example Alzheimer's Disease. Combination Therapy (Orally Available ActRIIB Activator with Neuronal Penetrance in Combination with an Orally Available ActRIIB Disinhibitor with Neuronal Penetrance, for Example a non Immunosuppressive Immunophilin).

A patient presents with symptoms indicative of Alzheimer's disease. An orally available AcRIIB agonist with neuronal penetrance is administered by mouth at a dose of 0.1 μg-10 mg/kg of body weight per day to the patient to prevent Alzheimer's associated neurodegeneration. Mechanisms used to identify small molecule, orally bioavailable ActRIIB agonists with neuronal penetrance have been mentioned previously. Treatment is commenced as soon as possible and frequently enough to maintain neuroprotective blood levels as would be determined. The patient also receives V-10,367 by mouth at a dose of 100-200 mg/kg per day 1 hour before oral administration of the ActRIIB agonist. A non immunosuppressive FKBP12 antagonist is preferred for long term treatment.

The invention, in its various aspects, is also illustrated by the following non-limiting experiments. All studies were approved by the University of Auckland Animal Ethics Committee; and all efforts were made during the course of the studies to minimize animal suffering and the number of animals used.

Experiment 1

Unilateral hypoxic-ischemic brain injury was induced in 21-day-old Wistar rats using a modified version of the “Levine” rat preparation as described previously (Sirimanne E. S., Guan J., Williams C. E., and Gluckman P. D. (1994). Two models for determining the mechanisms of damage and repair after hypoxic-ischemic injury in the developing rat brain. Journal of Neuroscience Methods, 55:7-14), (Vannucci, R. C. (1993) Experimental models of perinatal hypoxic-ischemic brain. APMIS 101, 89-95), (Levine (1960) Anoxic encephalopathy in rats. American Journal of Pathology 36, 1-17). Briefly, rats of both sexes weighing 40-49 g were anesthetized and maintained on a 2% halothane/oxygen mixture. A 10 mm incision was made along the midline of the neck exposing the right common carotid artery which was double ligated using 4-0 silk surgical thread. Following a one hour recovery period in an infant incubator kept at a stable thermoneutral environment (34° C., 85±5% humidity), the rats were subsequently exposed to severe inhalation hypoxia of 8% oxygen in nitrogen for 60 minutes, at 34° C. and 80% humidity. They were then removed from the incubator and held at room temperature of 22° C. and 55=5% relative humidity, and fed food and water ad libitum. Following surgical treatment and hypoxia, rats were euthanized with an overdose of pentobarbital at the required time point (0, 3, 5, 10, 24, 48, 72, 120, 168 hours). Brains were removed and immediately frozen at −80° C.

Two controls were used: control rats, which did not undergo either ligation or hypoxia; and “sham” ligated rats, which underwent anesthesia and incision (but not ligation) and hypoxia. Because the 24 hour time point was deemed to be important in terms of activin expression from previous literature (Wu, D. D., Lai, M., Hughes, P. E., Sirimanne, E., Gluckman, P. D. and Williams, C. E. (1999) Expression of the activin axis and neuronal rescue effects of recombinant activin A following hypoxic-ischemic brain injury in the infant rat. Brain Research 835, 369-78), (Lai, M., Sirimanne, E., Williams, C. E. and Gluckman, P. D. (1996) Sequential patterns of inhibin subunit gene expression following hypoxic-ischemic injury in the rat brain. Neuroscience 70, 1013-24), sham rats and control rats were euthanized at this time point before their brains were removed.

Frozen whole brains were weighed quickly before being cut in half longitudinally through the midline into left and right hemispheres. Each brain hemisphere was transferred to a 5 mL vial on ice; to which 1 volume of lysis buffer (0.05 M Tris-HCl (pH 6.8), 0.025 M sucrose, 1 mM EDTA, 1 g/L sodium azide, 1 μg/mL pepstatin A, 0.1 mg/mL phenyl methyl sulfonyl fluoride) was added, where 1 volume is equal to the mass of 1 brain hemisphere (1.21-1.54 g). Brain hemispheres were then homogenized twice for 15 seconds on ice using a Brinkman homogenizer (Brinkman Instruments Inc., Utah, USA), sonicated twice on ice for 15 seconds using a Branson Sonifier cell disrupter (Branson Ultrasonics Corp., CT, USA), then centrifuged at 10000×g for 7 minutes at 4° C. The supernatant was collected and transferred to a 1.5 mL Eppendorf tube which was stored at −80° C.

To assay the amount of total protein in each sample, a bicinchinonic acid (BCA) cellular protein assay was carried out. Bovine serum albumin (BSA), fraction V, GIBCO BRL (Life Technologies, Auckland, New Zealand) was diluted in 1 M sodium hydroxide to a concentration of 4 mg/mL. Serial 1:2 dilutions of this stock BSA standard were prepared for a standard curve (0-2000 μg/mL). A 96 well ELISA plate was used for the assay. In duplicate, 50 μL of standards were added to the first two columns of the plate. Samples were diluted 1:40 in NaOH before 50 μL aliquots of each were transferred to the plate in duplicate, starting from the top of the third column. 100 μL of BCA reagent was then added to each well and the plate incubated on an orbital plate shaker for 1-4 hours at room temperature. The plate was read at 562 nm on a Spectramax plate reader and computer, using SOFTMAX ProV software.

Total activin A, inhibin A and follistatin were measured by specific ELISAs as described previously (Keelan J A, M. K., Sato T A, McCowan L M, Coleman M, Evans L W, Groome N P, Mitchell M D (1999) Concentrations of activin A, inhibin A and follistatin in human amnion, choriodecidual and placental tissues at term and preterm. Journal of Endocrinology 163, 99-106). Brain homogenate for activin and inhibin ELISAs was diluted at 1:2.5 and 1:3 respectively, while follistatin ELISAs were carried out using neat homogenate. Data were collected and expressed as mean activin/inhibin/follistatin concentrations per mg protein and per mg tissue. Significance was calculated using a paired students t-test, with significance set at p<0.2.

SDS-PAGE on 12 per cent polyacrylamide gels under reducing conditions and transfer to PVDF membrane (Amersham Pharmacia Biotech, Buckinghamshire, UK) was carried out using a Mini Protean II/Trans-blot system (Bio-Rad, CA, USA) according to standard protocols. The final protein concentration of the samples did not exceed 40 μg/lane. Following electrophoresis, the membrane was blocked overnight in 5% non-fat milk in PBS at 4° C. Blocking buffer was then removed, and the blot oxidized in 3% H₂O₂ in PBS for 20 minutes to enhance the anitigenicity of the activin βA subunit (Keelan, J., Song, Y. and France, J. T. (1994) Comparative regulation of inhibin, activin and human chorionic gonadotropin production by placental trophoblast cells in culture. Placenta 15, 803-18). The blot was further washed in distilled water and then rinsed in a 5% non-fat milk/PBS solution. It was then incubated overnight at 4° C. with a rabbit polyclonal antiserum (anti-βA81-107) diluted 1:600 in 5% non-fat milk in PBS.

The blot was washed, then incubated with biotinylated anti-rabbit IgG (Sigma Chemical Co., Mo, USA) diluted 1:1000 in 10% non-fat milk in PBS/Tween for 1.5 hours at room temperature. After washing it was incubated for 1 hour with streptavadin-peroxidase complex (Amersham Pharmacia Biotech, Buckinghampshire, UK) diluted 1:2000 in wash buffer (PBS/Tween). This was followed by six washes in wash buffer (PBS/Tween) before rinsing with distilled water. After a final series of washes, the membrane was developed and visualized using chemiluminescence (ECL, Amersham Pharmacia Biotech, Buckinghampshire, UK). Different exposure times were tested and the signal was visualized on Kodak X-ray film using autoradiography.

Immunohistochemistry was carried out on paraffin embedded brain sections of 21 day old rats which had undergone severe hypoxic-ischemic injury. Following severe HI injury, brains were fixed by transcardial perfusion with 4% paraformaldehyde in PBS (pH 7.4), dehydrated, delipidated, and embedded in paraffin wax. Sections were cut at 7 μm using a microtome and deparaffinized in a xylol/ethanol gradient. Activin μA immunoreactivity was detected using the anti-μA81-107 polyclonal antibody [1:100] and visualized by peroxidase-DAB staining as described (Keelan, J., Song, Y. and France, J. T. (1994) Comparative regulation of inhibin, activin and human chorionic gonadotropin production by placental trophoblast cells in culture. Placenta 15, 803-18). The secondary antibody used was anti-rabbit IgG developed in goat (Sigma Chemical Co., MO, USA). Great care was taken to ensure that the slides with injured tissue and control tissue were exposed to the stain for the same length of time. Excess staining was then washed off, the slides were dehydrated in ethanol, air-dried, mounted in histamount and scanned with a Leica DMR Light Microscope (Leica Microsystems, Wetzlar, Germany).

Several experimental controls were conducted to validate the specificity of the primary antibody used in these immunohistochemical studies. Several tissue sections were carried through the immunohistochemistry process, but not incubated with primary antibody, to demonstrate that the staining seen was in fact due to the binding of antibody to activin A protein. Additionally, pre-absorption of the anti-βA81-107 polyclonal antibody with recombinant human activin at 10 times the concentration of primary antibody, resulted in no significant activin A staining.

Results

The effects of severe hypoxic ischemtic injury on the expression of activin, inhibin and follistatin proteins by examining results from the three ELISA assays, plotting picograms/mg of total brain protein and/mg of brain tissue versus time (hours) after injury. Comparisons between brain protein and tissue were made to assess any possibility of a general down-regulation of protein synthesis (brain protein content) following severe HI.

A detectable level of expression of all three proteins was found in sham rat brain. HI injury markedly increased the expression of activin but not inhibin or follistatin proteins within the first 3 days post injury. Activin protein levels were lowest immediately following hypoxic ischemic injury (0 hours) and then increased dramatically and transiently from 10 hours to 48 hours in both the injured and uninjured hemisphere of the brain. The highest average concentration of activin protein was observed 24 hours post HI in the ligated (stroke/injured) hemisphere (8.7 μg/mg tissue). In comparison, inhibin and follistatin concentrations were significantly lower at the 24-hour time point. However, at later time points, the levels of follistatin but not inhibin do increase significantly (p<0.2). Although there was only a weakly significant difference in activin protein between sham rats and 24 hour rats on the injured hemisphere (p<0.1), this level was greatly increased on the uninjured hemisphere (p<0.01).

The injured hemisphere appeared to have a much higher increase in activin protein concentration compared with the uninjured at the 24 hour time point, however this was also associated with a large standard error. In contrast to the large increases seen in activin levels in both hemispheres at 24 hours, the levels of inhibin, an active antagonist of activin function, did not show any significant increases 24 hours post HI. A relatively strongly significant difference is seen in inhibin protein levels between sham rats and 0 hour post HI rats (p>0.05), indicating the possibility of an initial down-regulation of inhibin following HI injury.

In contrast, there seems to be a delayed increase in follistatin levels 5-7 days post HI in both ligated and non-ligated hemispheres (p>0.15). Follistatin concentrations were generally expressed at 5-15 times higher than inhibin, but were still much lower then activin levels.

Western blot analysis of brain tissue 24 hours post HI injury (when the activin surge is maximal), produced bands of approximately 25 kDa, consistent with the reported molecular weight of the activin molecule in its physiologically active state as a homodimer composed of fully processed beta-proforms (βA) (Mason, A. J., Berkemeier, L. M., Schmelzer, C. H. and Schwall, R. H. (1989) Activin B: precursor sequences, genomic structure and in vitro activities. Molecular Endocrinology 3, 1352-8); Mason, A. J., Farnworth, P. G. and Sullivan, J. (1996) Characterization and determination of the biological activities of noncleavable high molecular weight forms of inbibin A and activin A. Molecular Endocrinology 10, 1055-65).

Two separate 24 hour post HI homogenate samples (ligated hemisphere) and 4 separate sham homogenate samples (ligated hemisphere) were used to run the gel. Also included in this blot were two samples of rh activin A protein, at 70 ng and 7 ng concentrations. Single bands between 20-29 kDa representing μAμA dimeric activin A were clearly visible in the 70 ng and 7 ng rh activin A lanes as well as in the 24 hour stroke sample lanes, although these were at a fainter level. In sharp contrast no immunoreactive bands at this molecular weight were seen in any of the four sham samples at the estimated MW of 20-29 kDa. Sham samples were blotted and probed with the same anti-βA subunit antibody. An estimation of the amount of activin present in these samples was made based on the density of these immunoreactive bands using Scion® image analysis software. Assuming that no activin is present in sham brains, and from extrapolation of these data, densitometric analysis suggested that approximately 1 ng activin protein was present in the 24 hour stroke lanes.

The three regions that were examined immunohistochemically were the cerebral cortex, dentate gyrus, and the CA1 region of the hippocampus. Control brains (no hypoxic or ischemic injury) were used for immunohistochemistry studies to establish a basal level of activin protein present. In control brains we found very few cortical or hippocampal cells showing positive activin-like immunolabelling. In contrast, in injured brain tissue (24 hours post HI) we found numerous pyramidal neurons staining positively for activin A. Significant morphological damage was observed within the cortex of the injured hemisphere 24 hours post hypoxia, in comparison to both control and uninjured/non-ligated hemisphere; and significant activin A positive immunostaining of cerebral cortical neurons was observed, with predominantly cytoplasmic staining seen.

Within the dentate gyrus cell layer, immunostaining for activin A was again found to be upregulated in cells on the injured hemisphere in comparison to the uninjured, as well as the control hemispheres. Strong expression of activin A immunostaining cells was seen in cells (presumably neurons) of the dentate gyrus of the injured hemisphere. However, cells (presumably neurons) within the dentate gyrus of the non-ligated hemisphere also showed some degree of staining.

A clear upregulation of activin A immunostaining in cells was also seen in the hippocampal pyramidal cell layer of the injured/ligated hemisphere, in comparison to the uninjured/non-ligated hemisphere. Higher magnification of the CA1 hippocampal region showed the localization of this immunostaining to be predominantly cytoplasmic rather than nuclear.

Conclusions

Results from this experiment demonstrate a role for activin A in the neuronal response to hypoxic ischemic injury. The high levels of activin A protein produced 24 hours post injury, coupled with low levels of its functional antagonists inhibin and follistatin, clearly shows that injury will lead to an endogenous neurotrophic/neuroprotective response in part mediated by activin A. The low level of expression of activin antagonists likely makes activin A more accessible to neurons in the injury recovery process. This study demonstrates that the dimeric βAβA form of activin is produced in response to HI injury (although other forms of activin may also be produced in the brain following injury) and that activin A protein is predominantly expressed in cortical, hippocampal and dentate granule cells (presumably neurons) of the injured hemisphere. The following experiment (Experiment 2) shows that these regions express high levels of ActRIIB nRNA following HI. The upregulation of ActRIIB in these brain regions in concert with increased production of bioactive activin protein lends support to the hypothesis that activin protein is expressed in high amounts following HI injury, and will bind to its receptors to initiate neurotrophic/neuroprotective signaling pathways in neurons.

Experiment 2

Subunit specific single-stranded deoxyribonucleic acid (DNA) oligonucleotide probes for the four possible rat activin receptor components and Smad-2 were obtained from GeneDetect.Com Ltd, Auckland, New Zealand (http://www.GeneDetect.com). The differential regulation of activin receptor components was investigated both constitutively in the non-lesioned and after three different types of brain injury, traumatic brain injury, hypoxic-ischemic injury, and unilateral quinolinic acid lesion of striatum. The qualitative in situ hybridization results were then converted into quantitative data using an image analysis protocol.

The probe sequences used were as follows:

Rat Alk-2; 48 mer, corresponding to base-pairs 687-735 in the Alk-2 sequence (Tsuchida, K., Mathews, L. S. and Vale, W. W. (1993) Cloning and characterization of a transmembrane serine kinase that acts as an activin type I receptor. Proceedings of the National Academy of Sciences of the United States of America 90, 11242-6):

| | |
|---|---|
| Antisense: | 5′ AAG ACC GGA GCC ACT TCC TGA TGT ACA |
| | TGA GTG ATC TAG TAA TTC CGC 3′; |
| Sense: | 5′ TTC TGG CCT CGG TGA AGG ACT ACA TGT |
| | ACT CAC TAG ATC ATT AAG GCG 3′. |

Rat Alk-4; 47 mer, corresponding to base pairs 472-519 in the Alk-4 sequence (Takumi, T., Moustakas, A., Lin, H. Y. and Lodish, H. F. (1995) Molecular charactetization of a type I serine-threonine kinase receptor for TGF-beta and activin in the rat pituitary tumor cell line GH3. Experimental Cell Research 216, 208-14):

| | |
|---|---|
| Antisense: | 5′ CAT GAG GGG TCC TCC ATG TCC AGT CTT |
| | TGG CGG TTG TGG TAG ACA CG 3′; |
| Sense: | 5′ GTA CTC CCC AGG AGG TAC AGG TCA GAA |
| | ACC GCC AAC ACC ATC TGT GC 3′. |

Rat ActRIIA; 48 mer, corresponding to base pairs 482-530 in the ActRIIA sequence (Shinozaki, H., Ito, I., Hasegawa, Y., Nakamura, K., Igarashi, S., Nakamura, M., Miyamoto, K., Eto, Y., Ibuki, Y. and Minegishi, T. (1992) Cloning and sequencing of a rat type II activin receptor. FEBS Letters 312, 53-6):

| | |
|---|---|
| Antisense: | 5′ GGA GGG TAG GCC ATC TTG TGA TGT CTG |
| | TAC ACC CAA AAC GCA CAA ATG 3′; |
| Sense: | 5′ CCT CCC ATC CGG TAG AAC ACT AGA GAC |
| | ATG TGG GTT TTG CGT CTT TAC 3′. |

Rat ActRIIB; 48 mer, corresponding to base pairs 1012-1060 in the ActRIIB sequence (Legerski, R., Zhou, X., Dresback, J., Eberspaecher, H., McKinney, S., Segarini, P. and de Crombrugghe, B. (1992) Molecular cloning and characterization of a novel rat activin receptor. Biochemical [symbol] Biophysical Research Communications 183, 672-9):

| | |
|---|---|
| Antisense: | 5′ GCA GCC AAA GCG ACC TCG AGC CTT GAT |
| | CTC CAG CAA CTG TAG CGC CTT 3′; |
| Sense: | 5′ CGT CGG TTT CGC TGG AGC TCG GAA CTA |
| | GAG GTC GTT GAC ATC GCC GAA 3′. |
| Smad2: | |
| Antisense: | 5′ ACA GTT GGG GCT CTG CAC AAA GAT CGC |
| | ACT ATC ACT TAG GCA CTC GGC 3′. |

To produce traumatic brain injury (TBI), male Wistar rats obtained from the Animal Resource Unit, Faculty of Medicine and Health Science, University of Auckland, and weighing between 300-400 g, were anesthetized with pentobarbital and positioned in a stereotaxic frame. A longitudinal incision was made in the overlying skin to expose the skull. A burr hole (1.5-2 mm) was drilled in the skull above the left cortex of the rat at co-ordinates AP-3.8, L 2.5 (bregma=0, mouth bar set at interaural (I/A) zero) using a standard dental drill and bit. The tip of a 50 μL Hamilton syringe was placed in the burr hole and then, over 3 min, was lowered 3 mm from the dural surface under stereotaxic guidance, positioning it in the underlying hippocampal formation. Coordinates for the injection were calculated using the rat brain atlas of Paxinos and Watson (Paxinos, G. and Watson, C. (1986) The Rat Brain in Stereotaxic Coordinates. Academic, Sydney). Five microlitres of saline was then injected into the hippocampus over 5 min (1 μL/min). At the end of the injection the syringe was carefully removed over 5 min to prevent reflux of injected solution into the needle tract. Once the syringe had been removed, the skin overlying the skull was sutured. Rats were euthanized by pentobarbital overdose at various times after brain injury; 30 min, 1 hr, 2 hr, 4 hr, 6 hr, 8 hr, 12 hr, 24 hr, and 48 hr (n=3 per timepoint). Three “sham” rats underwent all parts of the surgery but the intrahippocampal injection, hence they did not receive the focal hippocampal injury and were used to assign “basal” conditions. These rats were euthanized at 24 hr. The rat brains were rapidly removed and snap-frozen at −80° C. before coronal sections were cut at 16 μm on a Reichert-Jung cryostat and mounted on double-dipped, chrome-alum-coated microscope slides.

Unilateral severe HI injury was induced in 21 day old Wistar rats by the method described in Experiment 1; with groups of sham and control rats treated by that same method; and all rats euthanized as described in Experiment 1. Brains were then rapidly removed and snap-frozen at −80° C. before coronal sections were cut at 16 μm on a Reichert-Jung cryosrat and mounted on double-dipped, chrome-alum-coated microscope slides.

To produce QA lesioning of the striatum, male Wistar rats obtained from the Animal Resource Unit, Faculty of Medicine and Health Science, University of Auckland, and weighing between 300-400 g, were anesthetized with pentobarbital and positioned in a stereotaxic frame. A longitudinal incision was made in the overlying skin to expose the skull. Burr holes (1.5-2 mm) were drilled in the skull above the rats left (PBS) and right (PBS+QA) cortices at co-ordinates AP +0.5, L 3.0 (bregma=0, mouth bar set at co-ordinates −3.3 mm). For PBS infusion the tip of a 50 μl Hamilton syringe was placed in the left burr hole and then over 1 min was lowered 6.0 mm ventral from bregma, through neocortex to underlying striatum. Co-ordinates for the injection were calculated using the rat brain atlas of Paxinos and Watson. Then 2 μL of PBS (with no QA) was injected into striatum over 2 mnm (1.0 μL/min). Two minutes after the end of the injection the syringe was carefully removed over 1 min. Once the syringe was removed it was washed several times in PBS before being loaded with PBS+QA solution. In the same manner as before, 2 μL of PBS+QA solution was infused into the rat's right striatum and then the syringe was carefully removed. After removal, the skin overlying the skull was sutured. After surgery animals were left to recover in a quiet position.

The rats were euthanized at various times after QA lesioning; 3 hr, 5 hr, 10 hr, 24 hr, 2 day 3 day and 7 day (n=2 per timepoint); and two untreated rats were used as controls. Brains were then rapidly removed and snap-frozen at −80° C. before coronal sections were cut at 16 μm on a Reichert-Jung cryostat and mounted on double-dipped, chrome-alum-coated microscope slides.

In situ hybridization was used to visualize the differential expression of activin receptor component mRNAs and Smad2 in rat brain sections, and was performed using a standard protocol (Hughes, P., Beilharz, E., Gluckman, P. and Dragunow, M. (1993) Brain-derived neurotrophic factor is induced as an important immediate-early gene following N-methyl D-aspartate receptor activation. Neuroscience 57, 319-328; Hughes, P. and Dragunow, M. (1995) TrkC may be an inducible transcription factor target gene. Neuroreport 6, 465-468).

Further validation of the probes was done using the sense-oriented strands of the rat activin receptor components, and hybridizing antisense strands on RNase treated sections. The sense strand was hybridized to the coronal sections as per standard in situ protocol. A few amendments were made to the previously described protocol for the RNase treatment controls. RNase solution (200 mL) was made up as follows: 0.8 mL 10 mg/ml RNase, 4 mL 1 M Tris, 0.4 mL 0.5 M EDTA, distilled water to 200 mL.

Frozen cryostat cut coronal brain sections were fixed and washed by normal methods, and sections were either incubated with RNase solution or with RNase solution with no RNase enzyme added at 37° C. for 1 hr in a water bath. After incubation sections were washed (3×5 min, 0.1 M PBS) and then dehydrated by conventional methods. Sections were then handled using the usual in situ methods outlined previously to detect the rat activin receptor components.

Image analysis was performed on autoradiographs using the NIH Image analysis program, which measures the optical light density of scanned images. All autoradiographs were converted into digital images using a Canon Scanner, after which optical light density measurements were taken from specific regions where there was evidence of oligonucleotide probe binding. This data was then graphed and Student's t-test statistical analysis was performed using the Prism Graphpad program.

For the TBI model, both ipsilateral (injured) and the contralateral (uninjured) hemispheres were analyzed, and the measurements taken from the uninjured contralateral hemisphere were used as the internal control in the analysis procedure. We determined that no changes in gene expression occurred in the non-lesioned side of the brain in this model. Optical light density measurements were taken from the CA1 region, CA2 region, CA3 region, and dentate gyrus (DG); and two background measurements were also made in order to ensure consistency between autoradiograph results. The first was taken from within the hippocampus (BG1), and the second was taken from the ventral posteromedial thalamus (BG2). These measurements were taken from all the various time-points (n=3) and also from the shams (n=3) which acted as the major controls in this experiment.

The background measurement BG1 was subtracted from all the measured raw optical light density values (CA1, CA2, CA3, and DG) to give an “actual” value of the optical light density measurement in the respective hippocampal region. Hence, the measurements were adjusted for background. This was done for both the injured and uninjured hemispheres. The optical light density value for each region in the injured hemisphere was then converted into a percentage of the corresponding value in the uninjured hemisphere (internal control), hence giving a final value as a percentage of internal control. These were then averaged to give a final mean value (as a % control) for each respective region in each set of time-points, as well as sham. Hence; CA1−BG1=“actual” optical light density value for CA1. This was carried out for the CA1 region in both the injured and non-injured hemisphere. Then, the “actual” optical light density value for CA1 in injured (CA1 injured) was converted into a percentage of the corresponding value from the uninjured side (CA1 uninjured), using the equation CA1 mRNA expression (% Control)=(CA1 injured ÷CA1 uninjured)×100. Once the data was converted into a percentage, these were averaged for each set of time-points (n=3 for each). This average percentage value for each of the regions (CA1, CA2, CA3, and DG) per time-point were the final values used for graphing and statistical analysis in the Prism Graphpad program. Paired Student's t-test was used to test the statistical significance of any differential regulation of receptor mRNA levels.

For the unilateral severe HI model, both ipsilateral (ligated) and the contralateral (non-ligated) hemispheres were analyzed, and the measurements taken from the uninjured control rats were used as controls. Optical light density measurements were taken from: CA1 region, CA2 region, CA3 region, dentate gyrus DG), and parietal cortex (PC); and two background measurements (BG1 and BG2) taken as described above for the TBI model. The background measurement BG1 was subtracted from all the measured raw optical light density values (CA1, CA2, CA3, DG and PC) to give an “actual” value of the optical light density measurement in the respective hippocampal region. Hence, the measurements were adjusted for background. This was done for both the ipsilateral (ligated) and contralateral (non-ligated) hemispheres. The optical light density value for each region in both ligated and non-ligated hemispheres were averaged for each group of animals (n=3). The average value for each of the regions (CA1, CA2, CA3, DG and PC) per time-point were the final values used for graphing and statistical analysis in the Prism Graphpad program.

This analysis protocol was different from the one used for TBI for two major reasons. Firstly, the lack of an internal control. Unlike the TBI model, Unilateral HI model of brain injury does not have a internal control that can be used for the purposes of analysis. This is because both the ligated (ipsilateral) and non-ligated (contralateral) hemisphere seem to show changes in growth factor expression and, as previously mentioned, the changes seen on the contralateral side are due to seizure activity. And secondly, one of the receptors investigated showed no expression in the control group (optical density=0) and hence calculation of percentage control was impossible. This analysis protocol allowed both the non-ligated and ligated hemispheres of all the various post HI-insult time-points (and “shams”) to be compared to the control group. Paired Student's t-test was used to test the statistical significance of any differential regulation of receptor mRNA levels.

Results

Differential Regulation of Type I and Type II Activin Receptor mRNAs After Unilateral Traumatic Brain Injury

In “sham” adult and 21 day old rats, in situ hybridization revealed a homogenous expression of ActRIIA mRNA throughout the gray and white matter. A distinctly high level of expression was seen in the pyramidal neurons of the CA2 and the granular cells of the dentate gyrus. The amygdala region also had noticeable levels of ActRIIA mRNA expression. In adult animals undergoing TBI there was clear evidence of a transient unilateral downregulation of ActRIIA mRNA expression in the dentate gyrus of the injured hemisphere. The CA1, CA2 and CA3 regions did not show any change in ActRIIA mRNA expression with TBI. ActRIIA mRNA downregulation was evident at 6 hr, 8 hr and 12 hr post-injury. Paired Student' t-test revealed that the difference in ActRIIA mRNA expression between these time-points and the “sham” rats was statistically significant. ActRIIA mRNA expression was most significantly downregulated 8 hr post-injury (p<0.025). Time-points earlier than 6 hr or later than 12 hr post-injury did not show any differential regulation of ActRIIA mRNA within the injured DG, suggesting that ActRIIA expression was able to return to basal levels following injury.

In “sham” adult rats, in situ hybridization revealed very low but detectable levels of expression of ActRIIB mRNA throughout the brain. Unlike ActRIIA mRNA expression, ActRIIB mRNA was evenly expressed in the pyramidal neurons of CA1, CA2, CA3 and the granular cells of the dentate gyrus. This very low level of expression found in the adult brain contrasts with the almost complete absence of ActRIIB mRNA in the brain in the 21 day old rat. In adult rats there was no change in ActRIIB mRNA expression subsequent to TBI. The expression pattern of ActRIIB mRNA remained the same before and after injury for up to 48 hr, which was the last time-point examined in this experiment.

In “sham” rats, in situ hybridization revealed a homogenous expression of Alk-2 mRNA throughout the gray matter. The hippocampal formation including the pyramidal neurons of CA1, CA2, CA3 and dentate gyrus displayed the most notable labeling. Following TBI, there was clear evidence of a transient unilateral upregulation of Alk-2 mNA expression in the DG of the injured hemisphere. The comparison to the uninjured hippocampus. Alk-2 mRNA was significantly upregulated at 6 hr (p<0.025), 8 hr (p<0.10) and 12 hr (p<0.005) post-injury. Time-points earlier than 6 hr or later than 12 hr post-injury did not show any change in expression of Alk-2 mRNA in any brain regions on the injured side. Alk-2 mRNA expression was back to basal levels at 24 hr post-injury.

Alk-4 mRNA was the most widely expressed of all the receptor mRNAs investigated in the adult rat brain. The hippocampus shows a high level of Alk-4 mRNA expression which is evenly distributed in the CA1, CA2 and CA3 pyramidal layers and DG. Following TBI we observed no change in the levels of Alk-4 mRNA expression in the adult rat brain at any time point.

Sense probes for ActRIIA, ActRIIB, Alk-2 and Alk-4 probe as well as antisense probes for all four genes on RNase treated sections revealed no specific binding confirming the specificity of the subunit specific single-stranded DNA oligonucleotide probes.

Differential Regulation of Type I and Type II Activin Receptor mRNAs with Unilateral Hypoxic-Ischemic Brain Injury

In 21 day old “control” rats, in situ hybridization revealed a homogeneous expression of ActRIIA throughout the brain. The hippocampus showed clear expression of ActRIIA mRNA. A distinct “finger-print” expression pattern was see in the hippocampus with high levels of expression of ActRIIA mRNA in CA2 pyramidal cells and the granular cells of the dentate gyrus. CA1 and CA3 pyramidal cells had lower levels of ActRIIA mRNA expression. ActRIIA mRNA was strongly expressed in the amygdala as well as in the hippocampus. Unilateral HI injury had no observable effect on ActRIIA mRNA expression in the contralateral hemisphere. In contrast, the ligated hemisphere showed a significant but transient decrease in the expression of ActRIIA mRNA in certain brain regions compared to the control brain or contralateral side. There was transient downregulation of ActRIIA mRNA expression both in the dentate gyrus and CA2 pyramidal neurons at 3 and 5 hr post-injury. No change in ActRIIA mRNA expression occurred in any other region. A Paired Student's t-test revealed that the transient downregulation at 3 and 5 hrs following HI and ActRIIA mRNA expression in controls was statistically significant. “Sham” rats that underwent hypoxia-did not show any differential expression of ActRIIA mRNA.

In situ hybridization showed little or no detectable ActRIIB mRNA expression in the “control” 21 day old rat brain, which is in contrast to the low but detectable levels of ActRIIB mRNA seen in the adult rat hippocampus. However, following unilateral HI injury we found a dramatic rise in ActRIIB mRNA expression in the non-ligated hemisphere. Upregulation of ActRIIB mRNA was seen in CA1, CA2, and CA3 pyramidal neurons and the granular cells of the dentate gyrus of the hippocampus and in the overlying neocortex. The increase in ActRIIB mRNA expression was evident from as early as 3 hr post-injury and was still present as late as the seventh day post-injury, which was the latest time-point investigated in this study. Paired Student's t-test revealed that the upregulation of ActRIIB mRNA expression in the injured hemisphere was statistically significant. ActRIIB mRNA expression in the injured hippocampus peaked at 10 hr. At this time-point CA1 (p<0.005), CA2 (p<0.01) and DG (p<0.025) were at maximum levels; while CA3 (p<0.025) levels were fairly close to maximum, which was reached by the third day post-injury. There was also a significant increase in ActRIIB mRNA expression in the cerebral cortex of the ligated hemisphere. The parietal cortex displayed dramatic upregulation of ActRIIB mRNA expression from as early as 3 hr (p<0.01) post-injury, peaking at the 10 hr time-point (p<0.005) and significantly persisting through to the seventh day (p<0.005) post-injury, which was the latest time-point investigated in this study.

In the “control” 21 day old rat, in situ hybridization revealed an even distribution of Alk-2 mRNA expression throughout the coronal section with marked expression in the hippocampal formation. There was an even distribution of Alk-2 mRNA in CA1, CA2, and CA3 pyramidal neurons and the granular cells of the dentate gyrus. Both the injured and non-ligated hemispheres exhibited statistically significant (Student's t-test) upregulation of Alk-2 mRNA expression after HI injury. In the injured hemisphere there was a transient increase in Alk-2 mRNA expression at 24 hr post-injury in pyramidal neurons of the CA1 (p<0.025), CA2 (p<0.025), CA3 (p<0.05) layers and DG cell layer (p<0.05). This upregulation was not evident at any other time-point. In contrast, the non-ligated hemisphere displayed a rapid increase in Alk-2 mRNA expression. This rise in Alk-2 mRNA expression was evident only in the CA3 pyramidal neurons and cells of the DG and lasted until 5 hr post-injury. At the 10 hr time-point, expression was back to control levels. Then there appeared to be a second rise twelve hours later at the 24 hr time-point. The rise in Alk-2 mRNA expression at 24 hr was far greater in the DG than in the CA3 pyramidal layer.

Alk-4 mRNA was homogeneously expressed throughout the 21 day old rat brain in a similar distribution to Alk-2 mRNA, but was clearly expressed at higher levels than Alk-2. Indeed, of all four mRNA species, Alk-4 mRNA demonstrated the widest distribution and highest levels of expression throughout the “control” rat brain. In the hippocampus, CA1, CA2 and CA3 pyramidal neurons and granular cells of the DG expressed high levels of Alk-4 mRNA. In situ hybridization revealed no significant change in the expression of Alk-4 mRNA expression in the non-ligated hemisphere after HI injury, or in the ligated hemisphere within the first 24 hrs. In contrast, a dramatic fall in Alk-4 mRNA expression was seen in the CA1 and CA2 pyramidal layers of the hippocampus on the injured side 3 and 7 days post injury, possibly due to cell death. Paired Student's t-test revealed that this downregulation of Alk-4 mRNA in the pyramidal neurons of CA1/CA2 was statistically significant.

All four oligonucleotide probes were validated by hybridizing sense strands on tissue sections and antisense strands on RNase-treated sections. Both treatments revealed no specific signals confirming the specificity of the subunit specific single-stranded DNA oligonucleotide probes for the four activin receptor genes.

Differential Regulation of Type I and Type II Activin Receptor mRNAs after QA lesioning of the adult rat striatum

Both type I and type II ligand binding components exhibited a homogeneous expression of their respective mRNAs in the adult rat striatal sections. Expression of all four activin receptor component mRNAs was lightly distributed throughout the striatum including the frontal and parietal cortices.

There was no change in activin receptor mRNA expression post-QA lesioning. In order to test whether other related genes respond to this lesion paradigm, we examined the expression of the Smad2 transcription factor, which is an early intracellular downstream component of activin receptor signal transduction. Smad2 mRNA had a light distribution throughout the control adult rat striatum, very similar to the distribution of the activin receptor components. In situ hybridization revealed a pronounced upregulation of Smad2 mRNA expression at 24 hr which was maintained till the second day post-injury, demonstrating that the intracellular targets of ActRIIB signaling are present and respond to injury within the adult brain.

Conclusions

From this experiment we draw a number of conclusions. First, under basal non-injured conditions, the brain predominantly expresses the ActRIIA ligand binding receptor and that both type I receptors are also expressed throughout the nervous system. Following injury however the brain down-regulates the ActRIIA receptor, but up-regulates the expression of the ActRIIB receptor in regions of neuronal injury suggesting a role for this ActRIIB receptor in mediating the endogenous neuroprotective signals of the significant levels of dimetic activin A protein also expressed in these regions at these timepoints (Experiment 1). The concomitant low expression of inhibin and follistatin that can act as activin antagonists (Experiment 1) suggests that injury-induced activin will be bioactive and able to interact with ActRIIB receptors in injured brain tissue.

As exogenous administration of activin to the injured brain is neuroprotective (Hughes, P. E., Alexi, T., Williams, C. E., Clark, R. G. and Gluckman, P. D. (1999) Administration of recombinant human Activin-A has powerful neurotrophic effects on select striatal phenotypes in the quinolinic acid lesion model of Huntington's disease. Neuroscience 92, 197-209; Mattson, M. P. (2000) Activin to the rescue for overexcited neurons. Nature Medicine 6, 739-741; Tretter Y P, H. M., Munz B, Bruggencate G T, Werner S & Alzheimer C (2000) Induction of activin A is essential for the neuroprotective action of basic fibroblast growth factor in vivo. Nature Medicine 6, 812-815) we conclude that intracellular signals mediated by the ActRIIB receptor after injury are neuroprotective and that compounds that activate, or perhaps more importantly act to disinhibit the ActRIIB receptor subsequent to injury, have both neuroprotective and neurorestorative properties and may be used to provide neuroprotection and neurorestoration to injured, diseased, and degenerating neurons.

It will be appreciated by those persons skilled in the art that the above description is provided by way of example only and that numerous changes and variations can be made while still being with the scope of the invention as defined by the appended claims.

We claim:

1. A method of treating a mammal to rescue neurons otherwise destined to die, or to restore the phenotype and function of neurons degenerating, as the result of neuronal insult, comprising increasing within the mammal the concentration of an activin type IIB receptor-activating agent effective to rescue neurons otherwise destined to die, or to restore the phenotype and function of neurons degenerating, as the result of the neuronal insult.

2. The method of claim 1 where the activin type IIB receptor-activating agent is not activin.

3. The method of claim 1 where the activin type IIB receptor-activating agent is activin.

4. The method of claim 1 that is a method of rescuing neurons otherwise destined to die as the result of neuronal insult.

5. The method of claim 1 that is a method of restoring the phenotype and function of neurons degenerating as the result of the neuronal insult.

6. The method of claim 1 that comprises administering to the mammal a therapeutic amount of an activin type IIB receptor-activating agent.

7. The method of claim 6 where the activin type IIB receptor-activating agent is not activin.

8. The method of claim 6 where the activin type IIB receptor-activating agent selected from the group consisting of activins, activin agonists, and activin analogs.

9. The method of claim 8 where the activin type IIB receptor-activating agent is an activin.

10. The method of claim 9 where the activin is activin A.

11. The method of claim 8 where the activin type IIB receptor-activating agent is an activin analog.

12. The method of claim 8 where the activin type IIB receptor-activating agent is an activin agonist selected from the group consisting of bFGF, FSH, cAMP, TPA, TGF-β, IL-1β, and TNF-α.

13. The method of claim 9 that further comprises administration of an additional activin type IIB receptor-activating agent.

14. The method of claim 6 that further comprises administration of an agent that relieves inhibition of the activin type IIB receptor.

15. The method of claim 14 where the agent that relieves inhibition of the activin type IIB receptor is selected from the group consisting of follistatin, estradiol, FKBP12 antagonists, non-immunosuppressant orally bioavailable drugs, small molecules that disrupt TRAP-1 binding to neural type I activin receptors, small molecule antagonists of the mammalian version of the pseudoreceptor BAMBI, and agents which modulate endoglin and betaglycan function.

16. The method of claim 14 where the agent that relieves inhibition of the activin type IIB receptor is an FKBP12 antagonist selected from the group consisting of cyclosporin A, rapamycin, tacrolimus, ascomycin, GPI-1046, and N-Me-Val-4-cyclosporin A.

17. The method of claim 14 where the agent that relieves inhibition of the activin type IIB receptor is V-10,367.

18. A method of treating a mammal to rescue neurons otherwise destined to die, or to restore the phenotype and function of neurons degenerating, as the result of neuronal insult, comprising decreasing the active concentration of activin Type IIB receptor-inhibiting agents within the mammal to a level effective to rescue neurons otherwise destined to die, or to restore the phenotype and function of neurons degenerating, as the result of the neuronal insult.

19. A method of treating a mammal to rescue neurons otherwise destined to die, or to restore the phenotype and function of neurons degenerating, as the result of neuronal insult, comprising increasing within the mammal the concentration of an agent that relieves inhibition of the activin type IIB receptor effective to activate the activin type IIB receptor to rescue neurons otherwise destined to die, or to restore the phenotype and function of neurons degenerating, as the result of the neuronal insult.

20. The method of claim 19 that is a method of rescuing neurons otherwise destined to die as the result of neuronal insult.

21. The method of claim 19 that is a method of restoring the phenotype and function of neurons degenerating as the result of the neuronal insult.

22. The method of claim 19 that comprises administering to the mammal an amount of an agent that relieves inhibition of the activin type IIB receptor effective to activate the activin type IIB receptor.

23. The method of claim 22 where the agent that relieves inhibition of the activin type IIB receptor is selected from the group consisting of follistatin, ostradiol, FKBP12 antagonists, non-immunosuppressant orally bioavailable drugs, small molecules that disrupt TRAP-1 binding to neural type I activin receptors, small molecule antagonists of the mammalian version of the pseudoreceptor BAMBI, and agents which modulate endoglin and betaglycan function.

24. The method of claim 22 where the agent that relieves inhibition of the activin type IIB receptor is an FKBP12 antagonist selected from the group consisting of cyclosporin A, rapamycin, tacrolimus, ascomycin, GPI-1046, and N-Me-Val-4-cyclosporin A.

25. The method of claim 22 where the agent that relieves inhibition of the activin type IIB receptor is V-10,367.
